(19) United States
(12) Patent Application Publication
(10) Pub. No.: US 2020/0093623 A1
(43) Pub. Date: Mar. 26, 2020

(54) DEPLOYMENT HANDLE WITH STABILIZING SHELLS FOR A PRE-LOADED PROSTHESIS DELIVERY DEVICE

(71) Applicant: Cook Medical Technologies LLC, Bloomington, IN (US)

(72) Inventor: Kristoffer V. Bro, Aarhus N. (DK)

(73) Assignee: Cook Medical Technologies LLC, Bloomington, IN (US)

(21) Appl. No.: 16/575,859

(22) Filed: Sep. 19, 2019

Related U.S. Application Data
(60) Provisional application No. 62/734,361, filed on Sep. 21, 2018.

(51) Int. Cl. A61F 2/966; A61F 2/958

(52) U.S. Cl. CPC A61F 2/966; A61F 2002/9517; A61F 2/958

(57) ABSTRACT

A handle assembly for a prosthesis delivery device includes a proximal gripping portion, a stationary gripping portion, a shell assembly including first shell portion and a second shell portion extending distally from the proximal gripping portion to the stationary gripping portion when the proximal gripping portion is in a first position. The first shell portion and the second shell portion each have a part-cylindrical shape and complement each other to form a cylindrical shell assembly. Absent the first shell portion and the second shell portion being in the assembled state, the proximal gripping portion is distally movable toward the stationary gripping portion from the first position to a second position. The first position is a pre-deployment position and the second position is a deployment position. The first shell portion and second shell portion block the proximal gripping portion from moving distally from the first position into the second position.

### TECHNICAL FIELD

This invention relates generally to medical devices and methods of use, and more particularly, to an endovascular prosthesis delivery device handle and methods for placement and deployment of a prosthesis in the lumen of a vessel.

### BACKGROUND

An endovascular prosthesis, such as a stent, stent graft, vena cava filter or occlusion device, may be inserted into an anatomical vessel or duct for various purposes. For example, a stent graft may be delivered intraluminally from the femoral artery for treatment of vasculature in the human or animal body to bypass a repair or defect in the vasculature or to maintain or restore patency in a formerly blocked or constricted passageway. The stent graft may extend proximally and distally from a vascular defect, including a diseased portion of an aneurysm or dissection, and engage a healthy portion of a vessel wall.

The endovascular prosthesis to be implanted may be coupled to a delivery device in a compressed state and then released from the delivery device so as to expand within the vessel. The delivery device may then be withdrawn, leaving the endovascular prosthesis in position within the vessel. The steps to carry out the deployment of the endovascular prosthesis may occur in a pre-determined deployment sequence. For example, the delivery device may first be positioned within the vessel, and then the sheath retracted to allow the endovascular prosthesis to expand, at least partially. Further steps may then be performed, such as release of one or more trigger wires, for example, that facilitate release of one or both ends of the endovascular prosthesis, to deploy an anchoring stent, and the like. In most cases, it is desirable that such deployment steps follow a specific order as instructed by the manufacturer of the device.

Positioning and placement of the endovascular prosthesis, such as a stent graft, within a vessel lumen often requires the user to manipulate and rotate the handle of the delivery device to locate the endovascular prosthesis at a particular, pre-determined location during a procedure.

### SUMMARY

The present disclosure provides a handle assembly and a delivery system for delivering and deploying an endovascular graft.

According to an aspect of the present invention, there is provided a handle assembly for a prosthesis delivery device, the handle assembly comprising: a proximal gripping portion, a stationary gripping portion, a shell assembly including first shell portion and a second shell portion extending distally from the proximal gripping portion to the stationary gripping portion when the proximal gripping portion is in a first position, wherein the first shell portion and the second shell portion each have a part-cylindrical shape and complement each other to form a shell assembly having a cylindrical structure in an assembled state, wherein, absent the first shell portion and the second shell portion being in the assembled state, the proximal gripping portion is distally movable along a longitudinal axis toward the stationary gripping portion from the first position to a second position, wherein the first position is a pre-deployment position and the second position is a deployment position; and wherein the first shell portion and second shell portion block the proximal gripping portion from moving distally from the first position into the second position.

According to another aspect of the present invention, there is provided a delivery system for delivering a prosthesis, the delivery system comprising: an inner cannula having a proximal end and a distal end, wherein a prosthesis is releasably coupled to the proximal end of the inner cannula; a handle assembly disposed about the distal end of the inner cannula, the handle assembly comprising: a proximal gripping portion, a stationary gripping portion, and a shell assembly including first shell portion and a second shell portion extending distally from the proximal gripping portion to the stationary gripping portion when the proximal gripping portion is in a first position, the first shell portion and the second shell portion each having a part-cylindrical or part-prism shape and complement each other to form a shell assembly having a cylindrical or prism structure in an assembled state, wherein, absent the first shell portion and the second shell portion being in the assembled state, the proximal gripping portion is distally movable along a longitudinal axis toward the stationary gripping portion from the first position to a second position, wherein the first position is a pre-deployment position and the second position is a deployment position; and wherein the first shell portion and second shell portion block the proximal gripping portion from moving distally from the first position into the second position; and a sheath coupled to and extending proximally from the proximal gripping portion, wherein the sheath is disposed about the prosthesis when the proximal gripping portion is in the first pre-deployment position and wherein the sheath is retracted to expose at least a portion of the stent graft when the proximal gripping portion is in the second position.

Advantageously, the proximal gripping portion is adjacent to the stationary gripping portion when the proximal gripping portion is in the second position.

At least one of the first and second rails may include a cut-out providing visual access into the cylindrical structure formed by the first and second shells portions.

Each of the first and second shell portions preferably forms a semi-tubular rupture and includes a cut-out along a lateral edge, wherein the cut-out of the first shell portion longitudinally coincides and matches up with the cut-out of the second shell portion in the assembled state.

In preferred embodiments, the shell assembly comprises an outer surface with a grip-enhancing texture. The grip-enhancing texture may comprise longitudinal ribs.

Advantageously, the shell assembly operatively couples the stationary gripping portion to the proximal gripping portion. In particular, the shell assembly may engage the stationary gripping portion and the proximal gripping portion in a manner impeding relative rotation of the proximal gripping portion about the longitudinal axis relative to the stationary gripping portion. Preferably, the shell assembly engages the stationary gripping portion and the proximal gripping portion with contact surfaces providing high friction between the shell assembly and the proximal and stationary gripping portions. In at least some embodiments, the shell assembly engages the stationary gripping portion and the proximal gripping portion with contact surfaces providing a positive angular lock between the shell assembly and the proximal and stationary gripping portions.

Preferably, the shell assembly has one female end with a first contact surface and one male end with a second contact surface, the first contact surface being an exterior surface and the second contact surface being an interior surface. The first contact surface and the second contact surface may have longitudinal ridges complementary to further longitudinal ridges on the proximal and stationary gripping portions.

The handle assembly advantageously comprises at least one trigger wire release mechanism disposed about the longitudinal axis at a location distal to the stationary gripping portion. The at least one trigger wire release mechanism may be distally movable along the longitudinal axis when the proximal gripping portion is in the second position. A longitudinal movement of the at least one trigger wire release mechanism may be blocked when the proximal gripping portion is in the first position.

The teachings herein can provide a delivery device having a sturdy and stable handle, such that manipulation, rotation and movement of the handle at the distal end of the delivery device resists twisting and torque, resulting in the accurate corresponding movement of the endovascular prosthesis at the proximal end of the delivery device to ensure precise placement of the endovascular prosthesis within the vessel.

### BRIEF DESCRIPTION OF THE DRAWINGS

Embodiments of the present invention are described below, by way of example only, with reference to the accompanying drawings, in which:

FIG. 1 is a side perspective view of a prosthesis delivery device in pre-deployment configuration with an example of a handle assembly at the distal end and a nose cone dilator at the proximal end.

FIG. 2 is an exploded view of the prosthesis delivery device of FIG. 1.

FIG. 3 is a perspective view of a shell assembly being part of the handle assembly of FIG. 1.

FIG. 4 is a partial perspective view of the prosthesis delivery device of FIG. 1 after removal of the shell assembly in a configuration after sheath retraction and before trigger wire release.

FIG. 5 is a partial perspective view of the prosthesis delivery device of FIG. 1 with the distal-most trigger wire release mechanism sliding distally to deploy at least a portion of the prosthesis.

FIG. 6 is a partial perspective view of the prosthesis delivery device of FIG. 1 after all three trigger wire release mechanisms have been moved distally to deploy the prosthesis.

FIG. 7 is a partial exploded view of an alternative handle assembly.

### DETAILED DESCRIPTION

In this description, when referring to a prosthesis delivery device, proximal refers to the part of the delivery device that is furthest from the device operator and intended for insertion in a patient's body and distal refers to the part of the delivery device closest to the device operator. With regard to the prosthesis, the term proximal refers to that part of the prosthesis that is closest to the proximal end of the delivery device and distal refers to the opposite end of the prosthesis. Reference to coupling or connection of components, unless specified otherwise, includes direct connection as well as connection through intervening components.

In general and described in more detail below with reference to the reference numbers and Figures, the delivery device 2 includes a proximal end 4 and a distal end 6. A handle assembly 8 is located adjacent the distal end 6 of the delivery device 2.

The delivery device 2 including the handle assembly 8 are capable of use with a variety of prostheses. A prosthesis, such as a stent graft, may be disposed on a stent graft retention region 20 at the proximal end 4 of the delivery device 2 that terminates in a tapered nose cone dilator 22. The delivery device 2 including the handle assembly 8 is also suited for use with a variety of coupling methods for coupling a stent graft to the stent graft retention region 20, including, but not limited to, one or more trigger wires and diameter reducing ties, as would be understood by one of ordinary skill in the art.

The proximal end 4 of the delivery device 2 includes the stent graft retention region 20 and a tapered nose cone dilator 22. The nose cone dilator 22 has a proximal tip and a reverse distal taper at its distal end. The surface of the nose cone dilator 22 provides a smooth tapered surface to facilitate entry into and movement through a body vessel. The nose cone dilator 22 may include radiopaque material or be equipped with a radiopaque marker (not shown) to facilitate visualization of the nose cone dilator 22 in use.

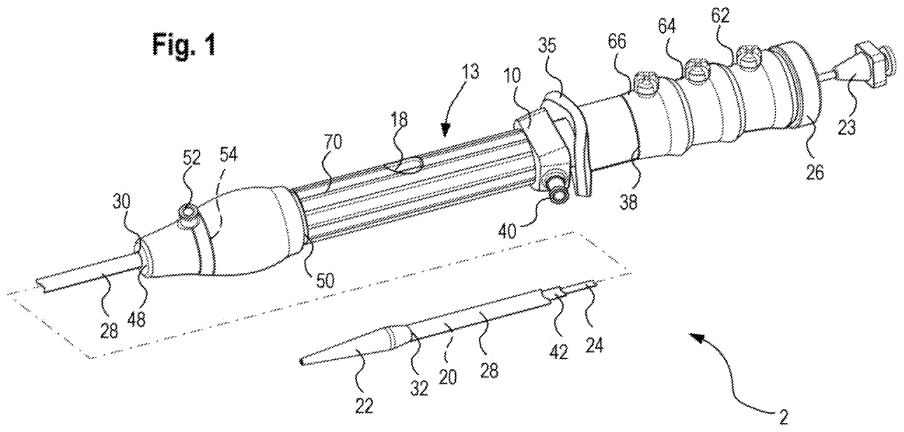

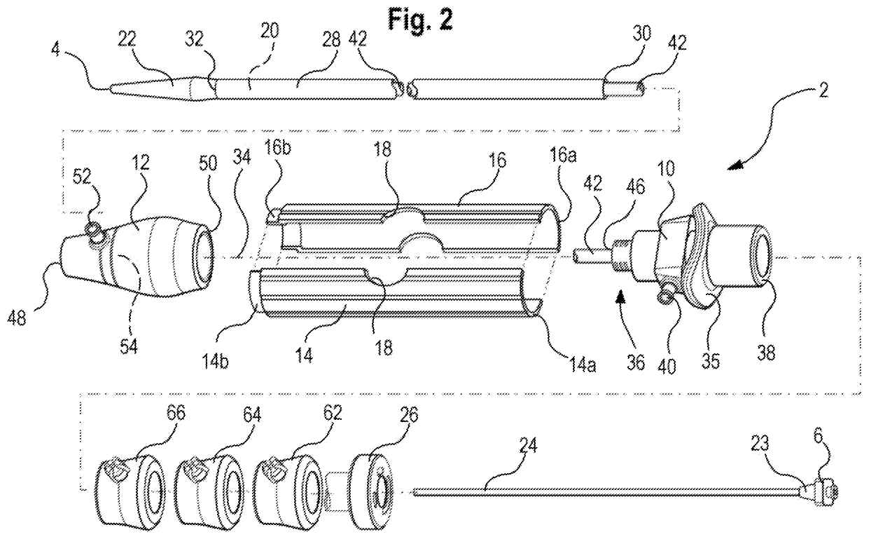

The delivery device 2 is shown in FIG. 1 in a pre-deployment configuration. As shown in more detail in a synopsis of FIG. 1 and FIG. 2, an inner cannula 24 extends along the longitudinal length of the delivery device 2, from a Luer adapter 23 at the distal end 6 of the delivery device 2 to the tapered nose cone dilator 22 at the proximal end 4 of the delivery device 2. The inner cannula 24 has an inner lumen which may accommodate a guide wire for tracking the delivery device 2 to a desired position within a patient's vasculature and which may be used for flushing or injection of fluids. The inner cannula 24 may be made of a variety of suitable materials including a flexible material, polymer, metal, or alloy, for example, nitinol or stainless steel, and may be either straight or have a curve imparted to a portion of it.

The handle assembly 8 shown in FIGS. 1 and 2 includes a stationary gripping portion 10 and a proximal gripping portion 12. The proximal gripping portion 12 is proximal to the stationary gripping portion 10 and is affixed to a sheath 28. A user may hold the stationary gripping portion 10 while manipulating the proximal gripping portion 12 (such as during sheath retraction during deployment). A distal longitudinal movement of the proximal gripping portion 12 causes the sheath 28 to retract distally from the nose cone dilator 22 and expose the stent graft retention region 20 for delivering a stent graft. As shown in FIG. 2, the stationary gripping portion 10 and the proximal gripping portion 12 define a central longitudinal axis 34.

Each of the stationary gripping portion 10 and the proximal gripping portion 12 may be ergonomically shaped for user comfort and may be covered in a layer of softer plastic or rubber or have a gripping surface to ensure a stable grip. Each of the stationary gripping portion 10 and the proximal gripping portion 12 may be injection molded as a single unitary structure, or alternatively, may be composed of parts that are connected by clam shell, lock, or snap fit, or are otherwise fixedly secured to each other.

A stiffener or positioner, referred to herein as a pusher catheter 42, extends proximally from the handle assembly 8. While omitted in FIG. 1 for simplicity, the pusher catheter 42 surrounds the cannula 24 and extends inside the sheath 28 (see FIG. 2. The pusher catheter 42 has a proximal end 44, a distal end 46, and a lumen extending from the proximal end 44 to the distal end 46, and is coaxial with the inner cannula 24. The distal end 46 of the pusher catheter 42 may be coupled to the stationary gripping portion 10, where it extends proximally through and beyond the proximal gripping portion 12 toward the stent graft retention region 20.

The stationary gripping portion 10 includes an irrigation port 40. This irrigation port 40 provides fluid communication with the pusher catheter 42 lumen. The pusher catheter 42 may be retained within the stationary gripping portion 10 by various means, including threaded attachment, adhesives, welding, or other suitable attachment mechanisms. The distal end 46 of the pusher catheter 42 may terminate within the housing provided by the stationary gripping portion 10. Additional stiffening materials or components, for example, a stiffening rod, (not shown) may be disposed over or used in combination with the inner cannula 24 or the pusher catheter 42 for additional stability and maneuverability when the delivery device 2 is in use.

The pusher catheter 42 may be constructed from various materials. In one example, a proximal portion of the pusher catheter 42 introduced into a patient may comprise a polymer, sometimes referred to as VRDT (or vinyl radiopaque dilator tubing), plastics, metals, alloys or a combination thereof. A distal portion of the pusher catheter 42 may comprise the same material as the proximal portion of the pusher catheter 42 or may be a different material, including but not limited to, plastics, polymers, alloys, metals or a combination thereof, that provide sufficient maneuverability and stiffness to the pusher catheter 42 as necessary and desired.

As shown generally in FIGS. 1 and 2, the sheath 28 has a proximal end 30, a distal end 32, and a lumen extending from the proximal end 30 to the distal end 32. In a pre-deployment configuration, the sheath 28 is configured to cover and assist in retaining a prosthesis (not shown), such as a stent graft, in a radially inwardly compressed, low-profile configuration during delivery of the prosthesis to a target site within a patient's anatomy. The sheath 28 extends proximally from the proximal end of the proximal gripping portion 12 to the nose cone dilator 22 at the proximal end 4 of the delivery device 2. The sheath 28 may be secured to the proximal gripping portion 12 by a friction fit, threaded engagement, adhesives, or other attachment mechanisms or combination thereof.

As generally shown in FIG. 1, a shell assembly 13 is initially positioned distally from the proximal gripping portion 12 and proximally from the stationary gripping portion, such that the rail assembly extends between the stationary gripping portion 10 and the proximal gripping portion 12 along a longitudinal axis 34 in a pre-deployment configuration. The shell assembly 13 blocks a distal movement of the proximal gripping portion 12 relative to the stationary gripping portion 10. The shell assembly 13 is further configured to impede relative rotation or torsion of the proximal gripping portion 12 about the longitudinal axis 34 relative to the stationary gripping portion 10. The shell assembly 13 is also configured to impede rotation of the handle assembly 8 about the longitudinal axis 34 relative to a prosthesis located at the proximal end 4 of the delivery device 2. The shell assembly 13 stiffens the connection between the proximal gripping portion 12 and the stationary gripping portion 10 and thus reduces torsion along the length of the delivery device 2, from the stationary gripping portion 10 of the handle assembly 8 to the distal end 4 of the delivery device 2.

In some embodiments, the shell assembly 13 is configured to facilitate a substantially 1:1 ratio of rotation between the stationary gripping portion 10, to which the pusher catheter 46 is affixed, and the proximal gripping portion 12 that in turn rotates the sheath 28. In other words, a particular degree of rotation of the stationary gripping portion 10, such as by rotation of the physician's wrist during use to properly position a prosthesis during a procedure, will result in the same degree of rotation of the prosthesis within the vessel lumen via the sheath 28. This provides precise control over positioning and placement of the prosthesis during a procedure. When a physician grasps the stationary gripping portion 10 and rotates the stationary gripping portion 10 to position the prosthesis at the proximal end of the delivery device 2 prior to sheath retraction, the shell assembly 13 serves as a stabilizing and reinforcing coupling between the stationary gripping portion 10 and the proximal gripping portion 12. Accordingly, the shell assembly 13 substantially reduces or prevents torsion and relative rotation between the stationary gripping portion 10 and the proximal gripping portion 12 when torque is applied to the stationary gripping portion 10. This also provides precise control over positioning and placement of the prosthesis while also preventing an inadvertent withdrawal of the sheath 28 and serving to prevent twisting along the length of the inner cannula 24, the sheath 28 and the pusher catheter 42.

In the example shown in the drawing figures, the shell assembly 13 includes two half shells, i.e. a first shell 14 and a second shell 16. Although the shown examples include two identical half shells 14 and 16, the half shells may differ from one another. The shapes of the half shells 14 and 16 complement each other and together form a tubular structure surrounding the pusher catheter 42 extending along the central axis 34 between the stationary gripping portion 10 and the proximal gripping portion 12. The shell assembly 13 shown has a cylindrical structure of substantially circular transverse cross-section. A circular transverse cross-section not essential, however. Other transverse shapes are possible, including, for example, oval and polygonal such as square or rectangular, or any other shape having at least three corners; in other words any prism.

The longitudinal axis 34 is at least substantially parallel to the half shells 14 and 16. The half shells 14 and 16 in FIG. 1 are arranged about, and parallel to, the longitudinal axis 34. The half shells 14 and 16 are also parallel to each other and in contact with each other on opposite lateral sides to form the tubular structure providing torsional stability. For visual access to the pusher catheter or any other structure within the tubular structure of the shell assembly 13, the half shells 14 and 16 are provided with cutout openings 18 extending through the shell to the inside of the tubular structure. In the shown example, the cutout openings 18 are formed by circumferential indentations of the lateral sides of each of the half shells. Each indentation of each of the half shells 14 and 16 matches with a corresponding indentation of the respective other one of the two half shells 14 and 16 such that the cutout openings of tubular structure of the shell assembly 13 extend across the boundary line between the two half shells 14 and 16. This arrangement of the cutout openings 18 also a free edge for prying the half shells 14 and 16 apart and thus facilitates the removal of the half shells 14 and 16 for enabling relative longitudinal movement of the proximal gripping portion 12 toward the stationary gripping portion 10. alternatively, the cutout openings may extend through the center of each of the two half shells 14 and 16, thereby providing visual access to the interior of the tubular structure of the shell assembly 13.

Additionally or alternatively, the half shells 14 and 16 may have a surface texture providing grip that facilitates a separation of the two half shells 14 and 16 from each other. In the shown example, such a surface texture is formed by longitudinal ribs 70 extending on the outer surface of each of the two half shells 14 and 16.

In one example, each of the half shells 14 and 16 has a female end 14a or 16a, respectively, and a male end 14b or 16b, respectively, as shown in FIGS. 2, 3, 4, and 7. The female end 14a, 16a, may include an axial extension dimensioned to mate with a section at the proximal end 36 of the stationary gripping portion, while the male end 14b, 16b, 18b may have one or more correspondingly shaped projections dimensioned to be inserted into the distal end 50 of the proximal gripping portion. Notably, the male and female ends may be reversed so that the shell assembly 13 may have the male ends 14b and 16b dimensioned to mate with a female portion of the stationary gripping portion and female ends 14a and 16a mate with a male portion of the proximal gripping portion.

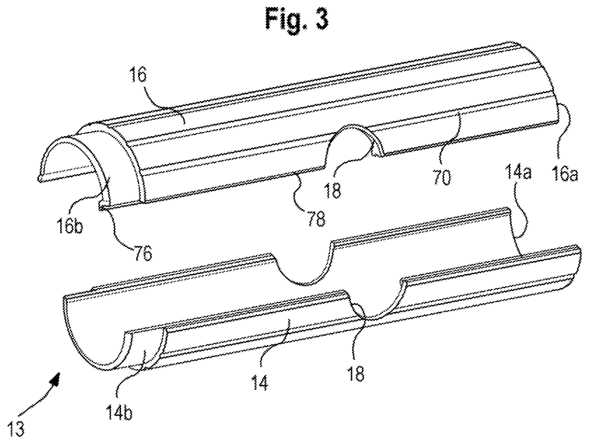

In the example shown in FIG. 2 and, in an enlarged view in FIG. 3, the shell assembly is configured to be coupled to the proximal gripping portion 12 and to the stationary gripping portion 10 by friction.

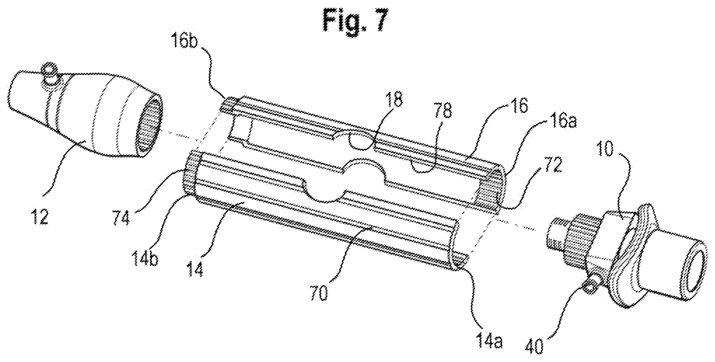

Alternatively, as shown in FIG. 7, the female ends 14a and 16a may have an internal contact surface with an inner spline 72 that meshes with a corresponding outer spline of the respective stationary or proximal gripping portion 10 or 12 to provide a positive angular lock between the shell assembly 13 and the male end of the respective gripping portion. In analogy, the male ends 14b and 16b may have an external contact surface with an outer spline 74 that meshes with a corresponding inner spline of the respective stationary or proximal gripping portion 10 or 12 to provide a positive angular lock between the shell assembly 13 and the female end of the respective gripping portion. While the internal and external splines 72 and 74 are shown as splines covering the entire circumference, different shapes are suitable, provided that the respectively mated male and female portions mesh with one another and impede angular relative movement between the respective surfaces contacting each other. In non-limiting examples, the contact surfaces may surround a non-circular cross-section, such as a square, rectangle, triangle, or hexagon, or may include a number of ribs meshing with grooves.

It is further possible for the shell assembly 13 to have opposing female ends and no male ends. At least in this case, it is preferred that the two half shells 14 and 16 are snapped together by a corresponding structure 76 along their edges 78 or held together in another releasable fashion so that the half shells 14 and 16 don't prematurely fall apart. Where one end of the shell assembly 13 is male and the other end is female, as shown in all embodiments of the drawings, the two half shells 14 and 16 may be sufficiently retained by the insertion of the male ends 14b and 16b into the respective female portion of the proximate or stationary gripping portion 12 or 10. Optionally, in this constellation, some additional releasable connection between the two half shells may be beneficial in a location closer to the female ends 14a and 16a than to the male ends 14b and 16b. The shell assembly 13 may also have two male ends. In this constellation, a slight proximal movement of the proximal gripping portion releases the half shells for removal.

During a procedure, the user may need to rotate the handle assembly 8 to adjust the position of a prosthesis within a vessel lumen. The handle assembly 8 may be rotated for example, by rotating the stationary gripping portion 10 or the proximal gripping portion 12. Because the half shells 14 and 16 couple the proximal gripping portion 12 to the stationary gripping portion 10, rotation of any portion of the handle assembly 8, such as the proximal gripping portion 12, ensures that the rotation of the remaining components of the handle assembly 8, such as the stationary gripping portion 10 is substantially the same as the rotation of the proximal gripping portion 12. In other words, substantially the same amount of torque applied to the stationary gripping portion 10 is transferred to the proximal gripping portion 12 by the half shells 14 and 16. Also, conversely, substantially the same amount of torque applied to the proximal gripping portion 12 is transferred to the stationary gripping portion 10 by the half shells 14 and 16.

Similarly, because the inner cannula 24 is disposed through the handle assembly 8 and the half shells 14 and 16 couple the proximal gripping portion 12 to the stationary gripping portion 10, rotation of any portion of the handle assembly 8, such as the proximal gripping portion 12, ensures that the rotation of the inner cannula 24 and prosthesis disposed on the stent graft retention region 20 of the inner cannula 24 is substantially the same as rotation of the handle assembly 8. In other words, substantially the same amount of torque applied to the proximal gripping portion 12 or the stationary gripping portion 10 of the handle assembly 8 is transferred to the inner cannula 24 so that torsion along the length of the delivery device 2 is minimized.

The stationary gripping portion 10 may comprise an ergonomic shape for the user to grasp securely and comfortably during use of the device. In one example, the stationary gripping portion 10 may include one or more projections or ribs made of soft or semi-soft rubber, plastic or other materials that provide a comfortable and secure gripping surface. In FIG. 1, the stationary gripping portion 10 includes a radially-outwardly extending portion, or flange 35. The flange 35 extends radially outward from the longitudinal axis 34. The flange 35 of FIG. 1 has a rectangular shape, but it may be formed as a variety of different shapes and sizes as long as the stationary gripping portion 10 is suitable for holding by a user. The stationary gripping portion 10 may also provide a housing for other components. For example, as shown in FIGS. 1 and 3, the stationary gripping portion 10 may include a fluid port 40 for introducing fluids to a patient during a procedure, for example through the pusher catheter 42. Fluids may also be drained or eliminated through this fluid port 40.

The proximal gripping portion 12 extends from the proximal end 48 to the distal end 50 with an outer surface or side wall to form a housing having an interior. A hemostatic valve 54 may be housed within the interior of the proximal gripping portion 12. In one example, the hemostatic valve 54 prevents the backflow of fluid within the delivery device 2. This includes any fluids introduced prior to a procedure or bodily fluids, including blood, from flowing back into the device during a procedure. The proximal gripping portion 12 may house additional mechanical components. The proximal gripping portion 12 may also include an irrigation port 52. This irrigation port 52 may provide fluid communication with the lumen of the sheath 28. The irrigation port 52 extending through the housing of the proximal gripping portion 12 may also be referred to as a sheath flush port. The irrigation port 52 may include a one way valve that communicates with the sheath 28 lumen to allow sheath 28 flushing prior to introduction into the vasculature.

The proximal gripping portion 12 is movable along the longitudinal axis 34 and movable relative to the stationary gripping portion 10. When the shell assembly 13 is removed, the proximal gripping portion 12 is movable distally along the longitudinal axis 34 toward the stationary gripping portion 10 during use from a first or proximal pre-delivery position (FIG. 1) to a second or distal delivery position (FIGS. 5-7) where the proximal gripping portion 12 is just proximal to and adjacent to the stationary gripping portion 10. With the proximal gripping portion 12 in this first or proximal position, the device is in a pre-deployment configuration. In this configuration, the device can be introduced into a patient's vasculature and manipulated by the user to a target site for vessel repair and for deployment of a prosthesis, such as a stent graft. In a pre-deployment configuration, a prosthesis carried at the proximal end of the device is covered by a sheath 28.

A rail 60 is affixed to the proximal gripping portion and extends distally to an end cap 26. The rail 60 extends through the stationary gripping portion 10 and optionally one or more trigger wire release knobs 62, 64, and 66 for aligning the stationary gripping portion 10 and the trigger wire release knobs 62, 64, and 66, where present, along the longitudinal axis 34.

The end cap 26 at the distal end of the rail 60 may be shaped as disc or ring and may serve as a stopping point for an object capable of sliding along one or more of the half shells 14 and 16 including, for example, the trigger wire release knobs 62, 64 and 66 where present. The disc-like shape of the end cap 26 is not intended to be limiting, and a variety of projection shapes and sizes may be suitable.

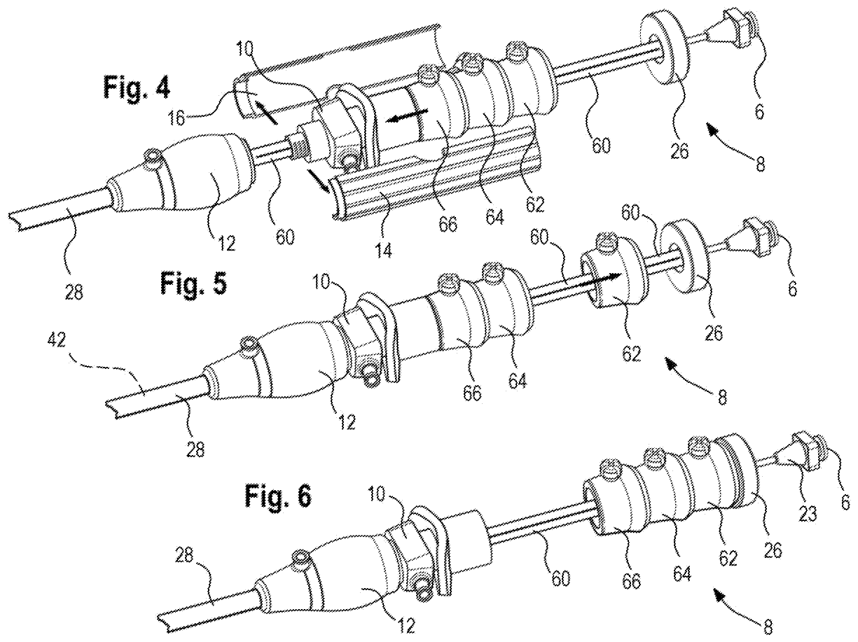

As shown in FIG. 4, the proximal gripping portion 12 is movable distally relative to the stationary gripping portion 10 when the half shells 14 and 16 are removed from the handle assembly 8. Due to the coupling between the proximal gripping portion 12 and the sheath 28, the distal longitudinal movement of the proximal gripping portion 12 retracts the sheath 28 distally along the longitudinal axis 34 when the proximal gripping portion 12 is retracted distally during deployment. By retracting the proximal gripping portion 12, the sheath 28 is retracted along with the proximal gripping portion 12 to uncover the stent graft positioned on the retention region 20. Once uncovered, the stent graft can be deployed by retraction and removal of the one or more trigger wires that retain the stent graft upon the inner cannula 24 at the stent graft retention region 20, as described herein.

When the proximal gripping portion 12 is moved to the second, distal-most delivery position as shown in FIG. 5, the sheath 28 is retracted as the proximal gripping portion is retracted, allowing the prosthesis retained at the proximal end 4 of the delivery device 2 to be deployed.

For example, once the sheath 28 has been retracted to uncover the prosthesis, deployment of the prosthesis may occur in one or more consecutive sequential steps, such as by the removal of one or more trigger wires, release of one or more diameter reducing ties or other mechanisms that retain the prosthesis on the delivery device 2 prior to deployment. In some situations, it may be desirable to deploy one end of the prosthesis prior to deployment of other parts of the prosthesis. The proximal end of the prosthesis may be deployed first, followed by deployment or release of other diameter-reducing ties at a one or more points along the length of the prosthesis followed by deployment of the distal end of the prosthesis. It is also contemplated that deployment of the graft can occur in a different sequence or by other mechanisms upon retraction of the sheath 28.

As previously mentioned, the stent graft release mechanism may include one or more trigger wire release mechanisms as needed for the sequential deployment of a prosthesis. While a plurality of trigger wire release mechanisms are shown and described herein in the form of trigger wire release knobs 62, 64, and 66 for the sequential deployment of a prosthesis, it will be appreciated that a variety of other methods and mechanisms may be suitable for use with the delivery device 2 to deploy a prosthesis.

An example of one suitable trigger wire release mechanism includes the trigger wire release knobs 62, 64, 66 as shown in FIGS. 4-6. A distal end of a trigger wire extending through the sheath 28 may be connected, attached or otherwise coupled to a trigger wire release knob. The trigger wire may extend proximally from the trigger wire release knob to a prosthesis carried at the proximal end of the device. The trigger wire may be coupled to the stent graft itself or to a diameter reducing tie or other mechanism that holds the prosthesis in a radially-inwardly constricted configuration prior to deployment. When the trigger wire is removed or withdrawn, the stent graft is released, allowing that portion or segment of the prosthesis to expand radially-outwardly and deploy. More than one trigger wire may be provided to allow the prosthesis to be deployed in stages and in a particular sequence.

In the example of FIGS. 4-6 three trigger wire release knobs 62, 64, and 66 are provided. The distal-most trigger wire release knob 62 is coupled to a trigger wire that may communicate with a portion of the prosthesis that needs to be released before other trigger wires are pulled. For example, the trigger wire release knob 62 may be coupled to a trigger wire controlling the release and deployment of the proximal end of the prosthesis. The middle trigger wire release knob 64 is coupled to another trigger wire that may communicate with the stent graft or a diameter reducing tie that is next in the order of deployment, for example along the length of the prosthesis, thus controlling the release and deployment of a central segment of the prosthesis. The proximal-most trigger wire release knob 66 is coupled to a third trigger wire that may communicate with a subsequently released portion of the stent graft, for example the distal-most end of the prosthesis and therefore controls the release and deployment of the distal end of the prosthesis. More or fewer trigger wire release knobs may be provided as necessary and desired depending on the number of trigger wires provided.

The trigger wire release knobs 62, 64, 66 have a range of longitudinal movement depending on several factors including, but not limited to, the length of the rail 60, the length of the stationary gripping portion 12, the number and lengths of trigger wire release knobs, and the position of the proximal gripping portion 12 relative to the stationary gripping portion 10.

In FIG. 1, which generally represents a pre-deployment configuration, the proximal gripping portion 12 is spaced proximally from the stationary gripping portion 10 by a pre-selected distance determined by the length L of a central portion the shells 14 and 16 between the female ends 14a and 16a and the male ends 14b and 16b, which overlap, respectively, with the proximal gripping portion 12 and the stationary gripping portion 10. This pre-selected distance may correspond generally to the length of the stent graft to be deployed, such that a distal longitudinal movement of the proximal gripping portion 12 toward the stationary gripping portion 10 provides an adequate travel to retract the sheath 28 and fully uncover the prosthesis as described below.

In this pre-deployment configuration, the shell assembly 13 extends between the stationary gripping portion 10 and the proximal gripping portion 12. In this position, the end cap 26 abuts the distal-most trigger wire release knob 62, such that all three of the trigger wire release knobs 62, 64, 66 are captured snugly between the end cap 26 and the distal end 38 of the stationary gripping portion 10. This prevents premature an unintended longitudinal movement of the trigger wire release knobs 62, 64, 66, thereby preventing premature release of the trigger wires from the prosthesis.

After removal of the shell assembly 13, a longitudinal movement of the proximal gripping portion 12 in the distal direction toward the stationary gripping portion 10 pushes the rail 60 longitudinally through the stationary gripping portion 10 as shown in FIG. 4. As the proximal gripping portion 12 is pulled distally toward the stationary gripping portion 10, the sheath 28 is also retracted distally to uncover at least a portion of the prosthesis at the proximal end of the device. The end cap 26 attached to the distal ends of the rail 60 moves distally along with the rail 60.

As shown in FIG. 5, with the proximal gripping portion 12 moved to its distal-most position and abutting the proximal end of the stationary gripping portion 10, the rail 60 extends distally (e.g. rearward) from the stationary gripping portion 10. In this position, there is now a length of the rail 60 available for the trigger wire release knobs 62, 64, 66 to slide distally upon when the user is ready to retract and release the trigger wires to sequentially deploy the graft. In other words, a length of the rail 60 becomes available and longitudinal movement of the trigger wire release knobs 62, 64, 66 upon the shell assembly 13 becomes available to release the prosthesis during deployment.

In embodiments with more than one trigger wire release knob, the multiple trigger wire release knobs 62, 64, 66 are sequentially movable to deploy side branches of a stent graft. As an example, after the sheath 28 has been at least partially retracted by sliding the proximal gripping portion 12 distally to a position as shown in FIG. 4 to expose at least a portion of the prosthesis, the user can then manipulate the trigger wire release knobs 62, 64, 66 individually and in this order to deploy the prosthesis.

In one example as shown in FIG. 5, the first trigger wire release knob 62 is distally slidable along the length of the rail 60 until the first trigger wire release knob 62 abuts the end cap 26. While FIG. 5 shows the proximal gripping portion in its distal-most position, it is evident from FIG. 4 that such a movement of the distal-most trigger wire release knob 62 is already possible when the proximal gripping portion 12 has been moved distally from the pre-deployment position of FIG. 1, but has not quite reached the distal-most position.

The second trigger wire release knob 64 is incapable of any distal longitudinal movement along the rail 60 until the first trigger wire release knob 62 is moved distally, because the second trigger wire release knob 64 is sandwiched between the first trigger wire release knob 62 and the third trigger wire release knob 66. For this reason, the trigger wires operatively connected to the second trigger wire release knob 64 are prevented from being inadvertently removed from the prosthesis before deployment of the proximal end of the prosthesis as described above. Only after the first trigger wire release knob 62 has been retracted to deploy the proximal end of the prosthesis, can the user longitudinally retract the second trigger wire release knob 64 by sliding it distally along the rail 60.

Distal longitudinal movement of the second trigger wire release knob 64 along the rail 60, can proceed, until the second trigger wire release knob 64 abuts the first trigger wire release knob 62. As the second trigger wire release knob 64 is moved longitudinally along the rail 60 in a distal direction, the trigger wires attached to or otherwise operatively connected to the second trigger wire release knob 64 is pulled back with the knob 64 in a distal direction, thus removing the trigger wires from distal end of the prosthesis. This allows the distal end of the prosthesis to expand and deploy.

The third trigger wire release knob 66 is not capable of distal longitudinal movement upon the rail 60 until the second trigger wire release knob 64 is retracted, because the third trigger wire release knob 66 is sandwiched between the second trigger wire release knob 64 and the stationary gripping portion 10. For this reason, the trigger wires operatively connected to the third trigger wire release knob 66 are prevented from being inadvertently removed from the prosthesis before deployment of the distal end of the prosthesis as described above. Only after the second trigger wire release knob 64 has been retracted to deploy the distal end of the prosthesis, the third trigger wire release knob 66 is distally slidable along the rail 60.

After the second trigger wire release knob 64 has been retracted, the third trigger wire release knob 66 is slidable longitudinally along the rail 60 in a distal direction until the third trigger wire release knob 66 abuts the second trigger wire release knob 64.

This length of the rail 60, and thus, the longitudinal distance available upon which the trigger wire release knobs 62, 64, 66 can be distally retracted, is modifiable to suit a particular procedure, a particular stent graft size and length as well as the length of the one or more trigger wires.

A variety of shell assembly lengths and rail lengths may be used in combination to provide customizable options for a user. For example, a longer shell assembly 12 and rail 60 may be used to extend the overall rail length to accommodate a longer stent graft, allow for further longitudinal movement of the trigger wire release knobs 62, 64, 66 in a distal direction during deployment or for accommodating a larger number of trigger wire release knobs when a longer handle assembly 8 is desirable or necessary depending on the particular procedure being performed.

FIG. 6 illustrates the three trigger wire release knobs 62, 64, 66 after they have been moved in a distal direction along the rail 60 with respect to their position in FIG. 4.

When deployment of a prosthesis is desired, removal of the trigger wires from the prosthesis by distal retraction of the trigger wire release knobs 62, 64, 66 allows the prosthesis to move from a radially-inwardly constrained delivery configuration to a radially-outwardly expanded configuration within a vessel. The trigger wire release knobs 62, 64, 66 cannot be retracted in order to release the prosthesis from a radially-inwardly constrained delivery configuration to an appropriate state of deployment until after the proximal gripping portion 12 has been retracted to withdraw the sheath 28 and thereby expose the prosthesis. This is because the rail 60 (only schematically shown in the drawings) has not moved to the distal-most position as shown in FIG. 6 until after the proximal gripping portion 12 has been retracted in a distal direction to withdraw the sheath 28, thereby moving the rail 60 distally backward to provide a longitudinal distance or length upon which the trigger wire release knobs 62, 64, 66 can slide distally. In other words, until the proximal gripping portion 12 is retracted distally to withdraw the sheath 28, the trigger wire release knobs 62, 64, 66 are sandwiched between the distal end 38 of the stationary gripping portion 10 and the end cap 26. This structural arrangement prevents premature and unintended release of the trigger wires until the sheath 28 has been retracted to expose the stent graft.

A prosthesis may be coupled in a generally known manner to the stent graft retention region 20 of the inner cannula 24 such as by one or more trigger wires and/or circumferential diameter-reducing ties for facilitating insertion of the proximal end 4 of the delivery device 2 to a target location within a vessel lumen. The sheath 28 is positioned over the stent graft prior to deployment to radially-inwardly restrain the prosthesis for delivery in a low-profile configuration to a target site within a patient's anatomy.

In a non-limiting example of a releasable stent-graft attachment mechanism, three trigger wires (not shown) extend from the handle assembly 8, within the pusher catheter 42, to a prosthesis located at the proximal end 4 of the delivery device 2. More particularly, the distal ends of the trigger wires are each coupled to a respective trigger wire release knobs 62, 64, 66. One or more of the other trigger wires may be coupled to a distal end of the stent graft while other trigger wires may be coupled to one or more diameter reducing ties disposed around a center region of the stent graft. Other suitable attachment methods or mechanisms may be used to removably attach the trigger wires to the stent graft as would be recognized by one of skill in the art.

The operation of the delivery device 2, and in particular, one non-limiting example of a deployment sequence using a handle assembly 8 of a delivery device 2, will be described below. In this example, use of a delivery device 2 will be described in reference to the implantation of a prosthesis (not shown) in one or more arteries extending distally from the aorta of a patient.

After an incision is made in the artery of the patient, the nose cone dilator 22 is inserted into the incision and the device 2 is tracked over a guide wire (not shown) and advanced to a desired location for placement of the prosthesis at the site of an aneurysm or other target site needing repair. A desired imaging modality (i.e., fluoroscopy, MRI, 3D or other imaging techniques) may be used for proper positioning at the target site. Using the desired imaging modality and one or more radiopaque markers (not shown) on the prosthesis, the user may ensure that the prosthesis is properly oriented. The sheath 28 is disposed over the prosthesis and extends proximally from the proximal gripping portion 12 up to at least the distal end of the nose cone dilator 22 during insertion and delivery to the target site.

The prosthesis at the proximal end 4 of the delivery device 2 is fully sheathed and held in a radially inwardly contracted condition. When the position of the device 2 is satisfactory, the user may partially withdraw the sheath 28. Any rotational movement of the nose cone dilator 22 for proper placement may be performed by rotating the stationary gripping portion. The shell assembly 13 ensures that the rotation of the stationary gripping portion 10 translates to the proximal gripping portion so that the sheath 28 and the nose cone dilator 22 are rotated synchronously. Any torsion due to the applied torque is thus minimized. Once the proximal end of the delivery device 2 has been properly positioned, the shell assembly is removed, and the sheath 28 is retracted by moving the proximal gripping portion 12 distally, toward the stationary gripping portion 10 along the longitudinal axis 34 to expose at least the proximal end of the prosthesis. Other visual or mechanical signals may also be present on the delivery device 2 and/or handle assembly 8 to indicate proper positioning of the prosthesis after the sheath 28 has been at least partially retracted. At this stage, the user continues to retract the sheath 28 by moving the proximal gripping portion 12 distally toward the stationary gripping portion 10 along the longitudinal axis 34 to expose at least a portion of the prosthesis distal to the already exposed proximal stent end.

Once the proximal gripping portion 12 has moved to its distal-most position where it is adjacent to and/or abuts the stationary gripping portion 10, the sheath 28 is therefore also fully retracted to expose the prosthesis. The rail 60 extends behind (distal to) the stationary gripping portion 10 to provide a sufficient distal travel distance for the trigger wire release knobs 62, 64, 66 to slide longitudinally upon, in a distal direction, during trigger wire release and removal.

At this stage, the user can retract the first trigger wire release knob 62 (the distal-most knob) along the half shells 14 and 16 in a distal directed until the first trigger wire release knob 62 reaches the end cap 26. Retraction of the first trigger wire release knob 62 serves to retract the first trigger wire and remove it from the proximal end of the prosthesis, thus facilitating deployment of the proximal end of the prosthesis. The user may then manipulate the second trigger wire release knob 64 by sliding it longitudinally along the rail 60 in a distal direction until the distal side of the second trigger wire release knob 64 abuts the proximal side of the first trigger wire release knob 62, or another predetermined stopping location. Retraction of the second trigger wire release knob 64 serves to retract the second trigger wire and remove it from the distal end of the prosthesis, thus facilitating deployment of the distal end of the prosthesis. The user can then manipulate the third trigger wire release knob 66 by sliding it longitudinally along the rail 60 in a distal direction until the distal side of the third trigger wire release knob 66 abuts the proximal side of the second trigger wire release knob 64, or another predetermined stopping point. Retraction of the third trigger wire release knob 66 serves to retract the third trigger wire and remove it from one or more diameter reducing ties as well as any additional attachment mechanisms, to allow the prosthesis to fully deploy within a vessel.

For this example, the delivery device 2 comprises three trigger wire release knobs 62, 64, 66, and three trigger wires, although it will be appreciated that more or fewer trigger wire release knobs and/or trigger wires may be used as necessary or desired, depending on the type or size of stent graft being used and the particular procedure being performed, along with other factors.

Following deployment, and with the prosthesis released from the stent graft retention region 20, the delivery device 2 is withdrawn distally. In some instances, if delivery of another stent graft is desired, a guide wire may be left in place, even after the delivery device 2 is removed from the vasculature, to facilitate tracking of another delivery device 2 for the delivery and deployment of another prosthesis and/or stent graft.

When removing the delivery device 2 from the patient, the user may optionally slide the proximal gripping portion 12 in a proximal direction, thus sliding the sheath 28 proximally and allowing the proximal end 30 of the sheath 28 to extend over all of, part of, or at least a distal portion of the nose cone dilator 22 of the device 2 so as to “hub” or “recapture” a portion of the nose cone 22 within the sheath 28. With at least a distal portion of the nose cone dilator 22 recaptured within the sheath 28, the delivery device 2 can be withdrawn from the patient's body to complete the procedure, thus having effectively and efficiently deployed a prosthesis into one or more vessels in order to treat and/or restore patency to such vessel(s).

While the shell assembly of the described examples has a cylindrical structure of substantially circular transverse cross-section, a circular transverse cross-section is preferred but not essential. Other transverse shapes are possible, including, for example, oval and polygonal such as square or rectangular, or any other shape having at least three corners; in other words any prism.

1. A handle assembly for a prosthesis delivery device, the handle assembly comprising:
a proximal gripping portion,
a stationary gripping portion,
a shell assembly including first shell portion and a second shell portion extending distally from the proximal gripping portion to the stationary gripping portion when the proximal gripping portion is in a first position,
wherein the first shell portion and the second shell each have a part-cylindrical shape and complement each other to form a shell assembly having a cylindrical structure in an assembled state,
wherein, absent the first shell portion and the second shell portion being in the assembled state, the proximal gripping portion is distally movable along a longitudinal axis toward the stationary gripping portion from the first position to a second position, wherein the first position is a pre-deployment position and the second position is a deployment position; and
wherein the first shell portion and second shell portion block the proximal gripping portion from moving distally from the first position into the second position.

2. The handle assembly of claim 1, wherein the proximal gripping portion is adjacent to the stationary gripping portion when the proximal gripping portion is in the second position.

3. The handle assembly of claim 1, wherein at least one of the first and second rails includes a cut-out providing visual access into the cylindrical structure formed by the first and second shell portions.

4. The handle assembly of claim 2, wherein the first and second shell portions form a first half shell and a second half shell, each of the first and second half shell including a cut-out along a lateral edge, wherein the cut-out of the first half shell longitudinally coincides and matches up with the cut-out of the second half shell in the assembled state.

5. The handle assembly of claim 1, wherein the shell assembly comprises an outer surface with a grip-enhancing texture.

6. The handle assembly of claim 5, wherein the grip-enhancing texture comprises longitudinal ribs.

7. The handle assembly of claim 1, wherein the shell assembly operatively couples the stationary gripping portion to the proximal gripping portion.

8. The handle assembly of claim 7, wherein the shell assembly engages the stationary gripping portion and the proximal gripping portion in a manner impeding relative rotation of the proximal gripping portion about the longitudinal axis relative to the stationary gripping portion.

9. The handle assembly of claim 8, wherein the shell assembly engages the stationary gripping portion and the proximal gripping portion with contact surfaces providing high friction between the shell assembly and the proximal and stationary gripping portions.

10. The handle assembly of claim 8, wherein the shell assembly engages the stationary gripping portion and the proximal gripping portion with contact surfaces providing a positive angular lock between the shell assembly and the proximal and stationary gripping portions.

11. The handle assembly of claim 8, wherein the shell assembly has one female end with a first contact surface and one male end with a second contact surface, the first contact surface being an exterior surface and the second contact surface being an interior surface.

12. The handle assembly of claim 11, wherein the first contact surface and the second contact surface have longitudinal ridges complementary to further longitudinal ridges on the proximal and stationary gripping portions.

13. The handle assembly of claim 1, further comprising at least one trigger wire release mechanism disposed about the longitudinal axis at a location distal to the stationary gripping portion.

14. The handle assembly of claim 13, wherein the at least one trigger wire release mechanism is distally movable along the longitudinal axis when the proximal gripping portion is in the second position.

15. The handle assembly of claim 13, wherein a longitudinal movement of the at least one trigger wire release mechanism is blocked when the proximal gripping portion is in the first position.

16. A delivery system for delivering a prosthesis, the delivery system comprising:
an inner cannula having a proximal end and a distal end, wherein a prosthesis is releasably coupled to the proximal end of the inner cannula;
a handle assembly disposed about the distal end of the inner cannula, the handle assembly comprising:
a proximal gripping portion,
a stationary gripping portion, and
a shell assembly including first shell portion and a second shell portion extending distally from the proximal gripping portion to the stationary gripping portion when the proximal gripping portion is in a first position, the first shell portion and the second shell portion each having a part-cylindrical shape and complement each other to form a shell assembly having a cylindrical structure in an assembled state, wherein, absent the first shell portion and the second shell portion being in the assembled state, the proximal gripping portion is distally movable along a longitudinal axis toward the stationary gripping portion from the first position to a second position, wherein the first position is a pre-deployment position and the second position is a deployment position; and wherein the first shell portion and second shell portion block the proximal gripping portion from moving distally from the first position into the second position; and
a sheath coupled to and extending proximally from the proximal gripping portion, wherein the sheath is disposed about the prosthesis when the proximal gripping portion is in the first pre-deployment position and wherein the sheath is retracted to expose at least a portion of the stent graft when the proximal gripping portion is in the second position.

17. The delivery system of claim 16, wherein the shell assembly engages the stationary gripping portion and the proximal gripping portion in a manner impeding relative rotation of the proximal gripping portion about the longitudinal axis relative to the stationary gripping portion.

18. The handle assembly of claim 17, wherein the shell assembly engages the stationary gripping portion and the proximal gripping portion with contact surfaces providing high friction between the shell assembly and the proximal and stationary gripping portions.

19. The handle assembly of claim 17, wherein the shell assembly engages the stationary gripping portion and the proximal gripping portion with contact surfaces providing a positive angular lock between the shell assembly and the proximal and stationary gripping portions.

20. The delivery system of claim 16, further comprising at least one trigger wire release mechanism disposed about the longitudinal axis at a location distal to the stationary gripping portion, wherein the at least one trigger wire release mechanism is distally movable along the longitudinal axis when the proximal gripping portion is in the second position and blocked when the proximal gripping portion is in the first position.
